(19) United States
(12) Patent Application Publication
(10) Pub. No.: US 2015/0257908 A1
(43) Pub. Date: Sep. 17, 2015

(54) INDWELLING BODY LUMEN EXPANDER

(71) Applicant: The Board of Trustees of the Leland Stanford Junior University, Palo Alto, CA (US)

(72) Inventors: Tiffany E. CHAO, Boston, MA (US); Nicholas R. DAMIANO, San Francisco, CA (US); Shreya MEHTA, San Francisco, CA (US); John P. WOOCK, Costa Mesa, CA (US)

(73) Assignee: The Board of Trustees of the Leland Stanford Junior University, Palo Alto, CA (US)

(21) Appl. No.: 14/656,402

(22) Filed: Mar. 12, 2015

Related U.S. Application Data
(60) Provisional application No. 61/953,212, filed on Mar. 14, 2014.
(60) Provisional application No. 62/035,826, filed on Aug. 11, 2014.

(51) Int. Cl. A61F 2/89; A61F 2/848; A61F 2/04

(52) U.S. Cl. CPC A61F 2/89; A61F 2/04; A61F 2/848; A61F 2002/047

(57) ABSTRACT

Indwelling body lumen expanders which allow for the maintenance and patency of a body lumen, such as the prostatic urethra to relieve urethral obstruction, are described. Because the one or more expanders are configured to be relatively larger in diameter than the diameter of the body lumen, the expanders may become invaginated into the lumen wall thus enabling the prostheses to avoid fluid exposure which in turn prevent the prostheses from becoming encrusted or calcified.

### CROSS-REFERENCE TO RELATED APPLICATIONS

This application claims the benefit of priority to U.S. Prov. Apps. 61/953,212 filed Mar. 14, 2014 and 62/035,826 filed Aug. 11, 2014, each of which is incorporated herein by reference in its entirety.

### FIELD OF THE INVENTION

The invention relates generally to an indwelling body lumen expander for providing patency to the body lumen. More specifically, the invention relates to one or more indwelling body lumen expanders which may be deployed within a urethral body lumen to provide relief from urinary retention from conditions such as benign prostatic hyperplasia. The expanders and methods described may also be useful in other applications that require expansion of anatomical lumens, e.g., blood vessels, gastrointestinal tract, respiratory tract, reproductive tract, ureters, etc.

### BACKGROUND OF THE INVENTION

Urinary retention occurs when a patient has difficulty or an inability to empty the bladder. There are several causes, but the most common cause in men is benign prostatic hyperplasia (BPH), although urinary retention can also be caused by mechanical blockages from prostate cancer, bladder cancer, urinary tract injury, benign or malignant urethral stricture, etc. Urinary retention is distressing for patients, who may experience urinary hesitancy, straining, weak flow, dripping, incomplete emptying, dysuria, nocturia, frequent urination, and/or incontinence. When a urinary blockage becomes severe, it can cause more serious consequences, including kidney failure or sepsis.

Benign prostatic hyperplasia, also known as prostate enlargement, is the most common cause of obstructive urinary retention. The walnut-sized prostate gland PR surrounds the urethra just below the bladder outlet in men, and is known to enlarge with increasing age, as shown in the illustration of FIG. 1A. The bladder outlet is shown at the bladder neck BN opening into the prostatic urethra PU which extends through the prostrate PR. The prostatic urethra extends into the membranous urethra MU and further into the bulbar urethra BU. The portion of the urethra extending through the penis is shown as the penile urethra PU which opens into the urethral opening UO at its terminal end.

As the prostate PR encroaches on the urethra, the prostate PR may begin to obstruct the flow of urine from the bladder BL causing discomfort to the patient. BPH typically develops in men older than forty and has increasing prevalence with age, for instance, 50% of men over fifty and 90% of men over eighty have histological evidence of BPH.

The gold standard treatments for drug-refractory moderate-to-severe BPH are surgical in nature and typically involve removing all or part of the prostate gland PR. Treatments include open, laparoscopic, and/or robotic prostatic resections including transurethral resection of the prostate (TURP) and laser prostatectomy (LAP). These procedures, performed under general anesthesia, can result in post-operative pain and prolonged catheterization and carry a high risk of sexual side effects. Less invasive energy therapies include transurethral radiofrequency needle ablation (TUNA), transurethral microwave thermotherapy (TUMT).

Less-invasive mechanical methods, such as prostatic stents (e.g., UroLume®, American Medical Systems, Inc., Minnetonka, Minn.) have been developed as well. Most stent designs involve a tube of metal or plastic that spans the prostatic urethra PU. However, these stents often become excessively epithelialized, encrusted, migrated, or calcified when exposed to the urine stream. This in turn leads to discomfort, inflammation, re-obstruction, and infection.

Accordingly, less-invasive methods and devices for treating conditions such as BPH which do not succumb to the deficiencies above are desirable.

### SUMMARY OF THE INVENTION

The deployment of the one or more expandable structures or scaffolds within the prostatic urethra may be used to maintain patency of the prostatic urethra against the enlarged prostate in order to enable a patient to urinate and thereby provide relief from urinary tract retention arising from conditions such as BPH. Such urethral expanding structures may be deployed within the prostatic urethra and left as an indwelling prosthesis. Moreover, one or more of the expanding structures may be deployed adjacent to one another depending upon the degree of collapse of the prostatic urethra as well as the size and length of the prostatic urethra as well.

Generally, such an apparatus for maintaining patency of a body lumen may comprise an expander structure defining an opening and configured for contact against a tissue lining of a body lumen, wherein the expander structure has a diameter which sized to be, e.g., 0% to 60% larger than a diameter of the body lumen, and wherein the expander structure has a height which is configured to be invaginated within the tissue lining of the body lumen while maintaining patency of the body lumen.

In use, one method for maintaining patency of a body lumen may generally comprise advancing the least one expander structure which defines an opening into proximity of a body lumen which is to be maintained in an open configuration, deploying the at least one expander structure against a tissue lining of the body lumen such that the at least one expander structure has a diameter in a deployed configuration which is between, e.g., 0% and 60% larger than the typical diameter of the body lumen. Once expanded, the method may further comprise maintaining a position of the at least one expander structure within the body lumen such that the at least one expander structure is partially or completely invaginated within the tissue lining of the body lumen while maintaining patency of the body lumen.

The various expander structures described are designed to expand the cross-sectional area of the prostatic urethra to allow urine drainage from the bladder. These expanders may be deployed to span the prostatic urethra and may be optionally anchored at their respective locations to prevent migration of the structure after deployment in the patient body. The deployed diameter of the expander structures may be configured to expand to a deployment diameter which is generally larger than that of the urethra.

The one or more expander structures may also optionally incorporate a tissue anchoring mechanism which allows for the securement of the deployed expander directly into the surrounding luminal tissue walls to prevent migration of the structures. Moreover, the expander structures may be designed to have a height (the length of the expander along the luminal tissue wall) and width (wall thickness) which are sufficiently thin enough to allow for the luminal tissue to invaginate the expanders at least partially or completely into the urethral wall. The thinness of the expanders may facilitate the enfolding and/or epithelialization of the urethral tissue wall at least partially or completely around the expanders so that the structures remain out of the urine stream and are thereby protected from calcification or encrustation by the passing urine stream.

Furthermore, the expanders may also optionally incorporate any number of surface textures or features such as openings along the surface of the expanders which may facilitate the invagination of the tissue wall and/or epithelialization. Moreover, the expanders may also optionally incorporate any number of eluting biologic agents, e.g., alpha-blockers, 5-alpha reductase inhibitors, phosphodiesterase-5 inhibitors, etc. which may inhibit further tissue growth. Alternatively, the expanders may also optionally incorporate any number of eluting biologic agents which block cell proliferation, e.g., paclitaxel, sirolimus, zirolimus, etc.

In other variations, the expander may be coated with or integrate a layer of material which is hydrophobic or superhydrophobic to further discourage fluid exposure of the expander structure to the urine.

Additionally, the expander may also optionally incorporate various mechanisms for emitting any number of forms of energy or the expanders may be used along with various forms of energy which may be administered separately from the expander. For instance, an expander may administer thermal, electrical, microwave, mechanical, etc. energy to the tissue prior to, during, or after deployment of the expander. The source of the energy may be controlled or delivered externally from the patient body, e.g., via ultrasound or magnetic induction.

Additionally, the expanders may be comprised of any number of biocompatible materials, e.g., metals such as Nitinol or stainless steel, plastics, polymers such as silicone, PET, PTFE, etc., etc., which may be utilized in any number of combinations either within a single expander or between several expanders utilized together. While the expander structure may remain within the tissue walls, portions of the expander or the entire expander structure may be optionally configured to biodegrade or bioabsorb over a specified period of time.

In the event that the expander is comprised of a shape memory alloy such as Nitinol, the expander may be configured to have a predetermined diameter and shape. Prior to and during deployment of the expander within the body lumen, the expander may have a collapsed low-profile configuration suitable for uninhibited delivery into the body lumen. Once the expander has been desirably positioned, the expander may be allowed to self-expand into its deployment configuration as it heats to body temperature, e.g., above 30° C. Alternatively, the expander may be actuated to expand into its deployed configuration where it may then fully expand into deployment. In yet additional variations, a heating or cooling element (e.g., heated or cooled liquid or gas, Peltier junction, etc.) may be deployed with a deployment instrument or separate from the expander to adjustably heat or cool the expander to allow for its re-configuration into its deployed configuration.

The urethral expanders may be configured in a number of different embodiments. With any of the different configurations, a single expander may be deployed within the lumen or several expanders (e.g., 2 to 20 expanders or more) may be deployed adjacent to one another. In other variations, 2 to 4 expanders may be deployed while in other variations, 3 expanders may be deployed. Alternatively, the expanders may be deployed to overlap with respect to adjacent expanders. These several expanders may be deployed in a sequential or connected manner within the prostatic urethra that facilitates an open passageway from the bladder to the membranous urethra.

One variation may be configured to be adjustably sized either prior to deployment or during deployment via an adjustable locking mechanism which may allow for the sliding release and securement of the free ends of the expander member. The locking mechanism with adjustable ends may allow for the diameter of the expander to be adjusted between a specified minimum and maximum length. The first end may define a receiving channel having a number of projections for receiving the second end which may define one or more complementary projections such that the second end may be adjustably slid into the first end to allow for a ratcheted adjustment and securement between the ends.

Another variation may include an expander which defines an opening therethrough. In this variation, the ends of the expander may be secured to one another via a pin or lock which may extend through openings defined in each of the ends. This variation may also incorporate one or more struts which may be coupled at a hub and extend through the opening to support the expanded configuration of the expander once deployed.

Optionally, the expander structure may also incorporate an anchoring mechanism to facilitate securement of the structure to the surrounding tissue and to prevent its migration when in use. While a single tissue anchor may be used, multiple anchors may also be utilized in a uniform or arbitrary pattern along the outer surface of the expander. Moreover, such anchors may be configured in any number of shapes or patterns or projections which help to prevent expander migration, e.g., hook-shaped, barb-shaped, V-shaped, etc. Additionally, the anchors may not only be configured to extend radially form the expander, they may be configured to extend in alternate directions as well. During delivery of the expander within the body lumen, the anchors may be configured to have a low profile to prevent inadvertent engagement with the tissue wall but when the expander is deployed against the tissue wall, the anchors may be reconfigured, e.g., rotated or repositioned, to face the vessel wall upon deployment. The anchors may also be composed of the same material as the expander or they may be composed of another biocompatible material. While the anchors are illustrated in this particular example, such anchors may be optionally incorporated into any of the other variations of expanders as described herein.

In yet another variation of an expander structure, the expander may be configured into a collapsible structure forming a pattern, e.g., a zig-zag, coiled, sinusoidal, or other pattern, defining an opening which may facilitate the collapse and expansion of the expander along its circumference. In this variation, the expander may be expanded to the desired diameter which may then resist its collapse.

Another variation of the expander structure may be formed by telescoping elements comprised of separate sections which may be assembled into any number of polygonal shapes (e.g., square, rectangular, circular, triangular, elliptical, etc.). The expander assembly may form an octagonal shape (although other shapes are possible) where individual receiving sections may form female receiving channels along one or both ends for translatably receiving sections which may form male ends for insertion into the corresponding female channels of alternating receiving sections. The connections between the male and female ends may be configured to be uni-directional (e.g., ratcheting mechanisms within the receiving sections) so as to provide an expander structure which may be expanded to a deployed shape against the tissue walls which then resists collapse. In another variation, the assembly may form a triangular shape having receiving sections between adjustable male structures. Additionally, the assemblies may include any number of anchoring aspects along the external surfaces of the structures (as described herein) to prevent migration of the structures after deployment.

In yet another variation, the expander may be configured into a helical or spiral configuration which defines a lumen through the structure. This expander may be comprised of a single unitary structure which is maintained in a low-profile configuration for delivery within the urethra. Once deployed, the expander may reconfigure into its helical or spiral configuration against the tissue walls. Materials such as superelastic or shape memory alloys like Nitinol may be suitable for such a structure although other materials (as described herein) may also be utilized.

Other variations may include a single expander structure formed of separate ring-like structures which are aligned with respect to one another to define a lumen and which are coupled to one another via connector elements. The structure may be formed of a wire, ribbon, or other similar structure. However, the ring-like structures may be angled in a non-parallel manner with respect to one another in other variations. The ring-like structures may be formed as individual structures which are then connected to the connector elements although in other variations, the entire structure may be formed of a single element.

Moreover, the structure may include three ring-like structures for positioning along each of the three main lobes of the prostate so that the ring-like structures may become invaginated within the tissues of their respective lobes. Although alternative variations may include a structure having fewer than three or more than three ring-like structures as well as structures which are non-circular in shape but which may include any number of other shapes, as described herein. Other variations may be formed by a single structure which also connects each of the ring-like structures where the ring-like structures are formed by looping the element. Another variation may include connector elements which are parallel to one another but positioned along opposite ends of the ring-like structures. In alternative variations, the connector elements may be positioned at other angles relative to one another instead of being positioned at opposite ends.

Moreover, the relative spacing between the structures may also be varied and the structures themselves may be angled with respect to one another as well. Additionally, any of the structures may optionally incorporate any of the surface modifications such as anchoring mechanisms or various coatings or coverings, as described herein, in any number of various combinations.

In deploying any of the various expander assemblies described herein, various instruments may be employed. One example may include a delivery and deployment instrument having an elongate shaft having a diameter and a length suitable for insertion into the urethra of the patient. The shaft may be formed with a rigid length or partially flexible length so long as the shaft may be advanced, e.g., within the urethral opening and at least partially into the prostatic urethra. The shaft may have an inflatable member such as a balloon positioned near or at the distal end of the shaft which may have one or more expanders positioned upon the balloon in a low profile for intra-luminal delivery. Once the balloon has been suitable positioned within the body lumen, the balloon may be expanded to deploy the one or more expanders into contact against the lumen wall. Once deployed, the balloon may be collapsed for removal from the patient body. Another variation of a shaft may have an umbrella-like expansion mechanism for deploying one or more expanders against the tissue walls. Yet another variation of a deployment instrument may include an inflation reservoir and a tubular expander which may support one or more expanders upon the outer surface of the expander.

The deployment instrument may be advanced and positioned within the body lumen using an intraluminal visualization system, for example, an endoscope or cystoscope which may be integrated with the deployment instrument or separated. The visualization system may be utilized either before or during the deployment procedure. In other variations, an external imaging modality (e.g., ultrasound, computed tomography, magnetic resonance imaging, etc.) may be used in combination with the deployment instrument (and/or intraluminal visualization system). In some variations, the device may be inserted through the working channel of a rigid or flexible cystoscope or cystoscopy sheath, or through channels in other intraluminal imaging tools. In other variations, a balloon or other positioning reference system may be placed, e.g., within the bladder or elsewhere to ensure proper anatomic positioning of the expanders.

The system may also optionally include apparatus and methods for adjustment and/or retrieval of the one or more expanders. One embodiment of this aspect of the system may include a grasper mechanism (e.g., standard grasper tools may be used). Other variations may also include heating or cooling elements to adjust the device to facilitate adjustment and/or removal.

### BRIEF DESCRIPTION OF THE DRAWINGS

FIG. 1A shows an illustration of the urethral anatomy.

FIG. 1B shows an example of one variation of the expanders described herein deployed within the prostatic urethra.

FIG. 1C shows an end view of a single expander having multiple, e.g., three, ring-like structures or coils deployed against the tissue walls of the prostatic urethra.

FIG. 2 shows a top view of one variation of an expander having adjustable ends for sizing the expander to a desired diameter.

FIG. 3 shows a top view of another variation of an expander having a ratcheting mechanism for adjusting the diameter of the expander.

FIGS. 4A and 4B show top and perspective views of another variation of an expander having a pin/pinhole mechanism for adjusting the diameter of the expander.

FIGS. 5A and 5B show top and perspective views of another variation of an expander having a tissue anchoring mechanism.

FIG. 6 shows a perspective view of another variation of an expander configured to have an adjustably collapsible structure.

FIGS. 7A and 7B show top views of expanders having a structure forming a polygonal shape comprised of separate sections which may be adjustably telescoping relative to one another.

FIG. 8 shows a perspective view of an expander formed of a single element having a spiral or helical configuration.

FIG. 9A shows a perspective view of another variation of one or more expanders which are connected by a single spine element.

FIG. 9B shows a perspective view of another variation of one or more expanders formed of a single contiguous element which also forms a single spine.

FIG. 9C shows a perspective view of another variation of one or more expanders connected by spine elements which are aligned in an alternating manner relative to one another.

FIG. 10 shows a perspective view of another variation of one or more expanders which are connected to one another by multiple spine elements aligned in parallel.

FIG. 11 shows a perspective view of another variation of an expander having two ring-like structures or coils connected by a connector element which is longitudinally aligned.

FIG. 12 shows a perspective view of another variation of an expanding having two ring-like structures or coils connected by a curved connector element.

FIG. 13 shows a perspective view of the expander assembly of FIG. 11 deployed adjacent to a second expander assembly within the prostatic urethra.

FIG. 14A shows one variation of a deployment instrument having an inflatable structure such as a balloon for deploying and sizing the expanders.

FIG. 14B shows another variation of a deployment instrument having an umbrella-like expanding mechanism.

FIG. 15 shows another variation of a deployment instrument having an inflation expanding mechanism.

FIG. 16 shows a perspective view of an expander being deployed from a delivery instrument.

### DETAILED DESCRIPTION OF THE INVENTION

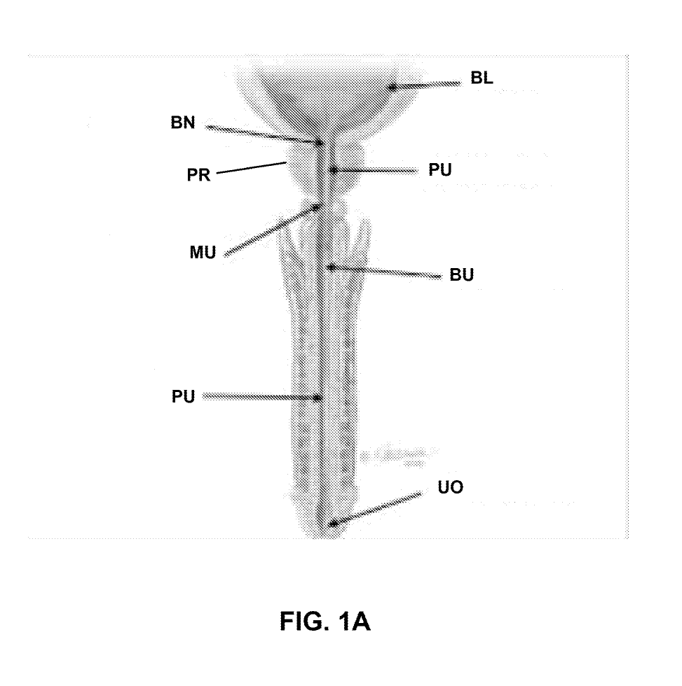

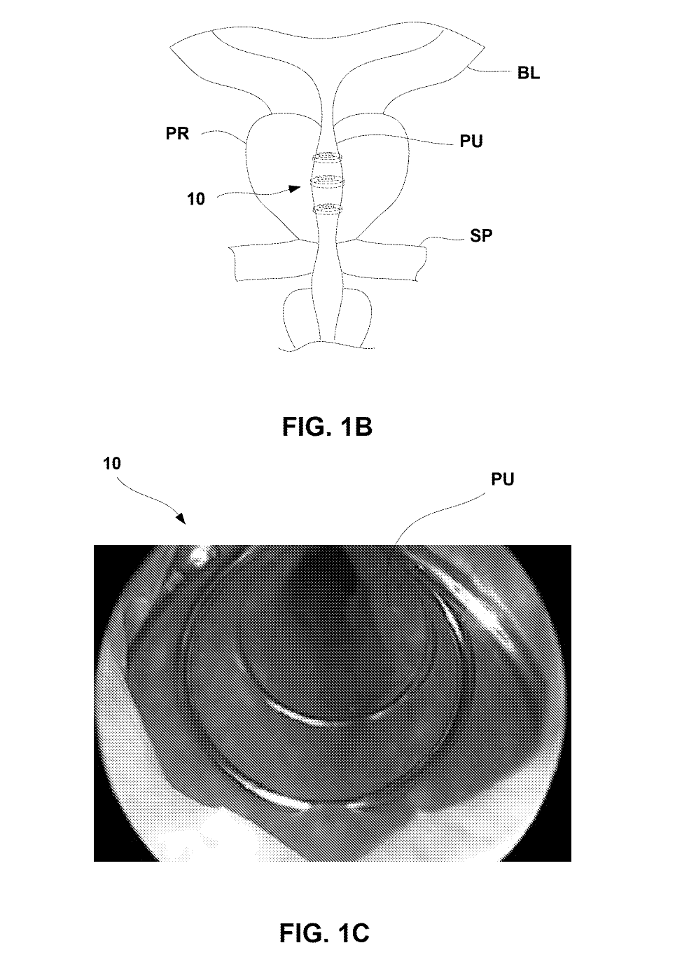

The deployment of one or more expandable structures or scaffolds within the prostatic urethra PU may be used to maintain patency of the prostatic urethra PU against the enlarged prostate PR (shown superior to the sphincter SP) in order to enable a patient to urinate and thereby provide relief from urinary tract retention arising from conditions such as BPH. Such urethral expanding structures 10 may be deployed within the prostatic urethra PU and left as an indwelling prosthesis, as shown in the side view of FIG. 1B. Moreover, one or more of the expanding structures 10 may be deployed adjacent to one another depending upon the degree of collapse of the prostatic urethra PU as well as the size and length of the prostatic urethra PU as well, as described herein.

FIG. 1C shows an end view of another example of an expanding structure 10 having three ring-like structures or coils deployed against the interior walls of the prostatic urethra PU. Although a single expanding structure 10 is shown, such a structure 10 may have fewer than three or more than three ring-like structures or coils, e.g., two to four, for deployment. Moreover, multiple expanding structures 10 may be deployed within a single urethra, as further described herein.

The description of certain variations of the expanders are not intended to limit the scope of the present invention but other examples, features, aspects, embodiments, and advantages of the invention will become apparent to those skilled in the art from the description herein. Moreover, the expanders and their uses are capable of different and obvious aspects, all without departing from the scope of the invention. Accordingly, the drawings and descriptions should be regarded as illustrative in nature and not restrictive.

The various expander structures described are designed to expand the cross-sectional area of the prostatic urethra PU to allow urine drainage from the bladder BL. These expanders may be deployed to span the prostatic urethra PU and may be optionally anchored at their respective locations to prevent migration of the structure after deployment in the patient body. The deployed diameter of the expander structures may be configured to expand to a deployment diameter which is generally larger than that of the urethra, e.g., the deployed diameter may have a diameter which is, e.g., 0% to 60% or more preferably 10% to 40%, relatively larger than the typical diameter of a prostatic urethra PU. While the anatomy of individual patients will vary, the deployment diameter of the expander structure may range anywhere from, e.g., 8 mm to 16 mm. Alternatively, the diameter of a patient's prostatic urethra PU may be taken prior to delivery of the expanders and one or more of the expander structures having a diameter which is correspondingly larger in size than the patient's body lumen may be selected for deployment.

The one or more expander structures may also optionally incorporate a tissue anchoring mechanism which allows for the securement of the deployed expander directly into the surrounding luminal tissue walls to prevent migration of the structures. Moreover, the expander structures may be designed to have a height (the length of the expander along the luminal tissue wall) and width (wall thickness) which are sufficiently thin enough to allow for the luminal tissue to invaginate the expanders at least partially or completely into the urethral wall. The thinness of the expanders may facilitate the enfolding of the urethral tissue wall at least partially or completely around the expanders so that the structures remain out of the urine stream and are thereby protected from calcification or encrustation by the passing urine stream. For example, an expander may have a height ranging anywhere from, e.g., 0.2 mm to 1.5 mm, and a width ranging anywhere from, e.g., 0.2 mm to 1.5 mm, so long as the dimensions of the expander facilitate the eventual partial or complete invagination of the expander within the urethral tissue wall. Once one or more expanders have been deployed, partial or complete tissue epithelialization may take anywhere from, e.g., 2 to 8 weeks, given the size of the expanders and the nature of tissue along the urethral wall. The illustration shown in FIG. 1B shows an example of the one or more expanders 10 which have been deployed within the prostatic urethra PU of a patient body. The one or more expanders 10 may be seen as having been partially or completely invaginated within the tissue walls of the urethra such that the expanders 10 are no longer in contact with the passing urine but still provide for patency of the prostatic urethra PU.

Furthermore, the expanders may also optionally incorporate any number of surface textures or features such as openings along the surface of the expanders which may facilitate the invagination of the tissue wall. For instance, the surface of the expander may be electropolished to decrease friction of the expander during deployment from a delivery instrument. Alternatively, the surface of the expander could also be roughened or otherwise altered to increase friction and anchoring ability in the urethra. In yet another alternative, portions of the expander may be roughened, e.g., the outer surfaces which contact the tissue walls, while the remainder of the surfaces may remain smooth to facilitate delivery and deployment. Roughening the device in this manner could also serve to promote anchoring in the urethra or epithelialization into the urethral wall.

Moreover, the expanders may also optionally incorporate any number of eluting biologic agents, e.g., alpha-blockers, 5-alpha reductase inhibitors, phosphodiesterase-5 inhibitors, etc. which may prohibit additional prostate growth. Alternatively, the expanders may also optionally incorporate any number of eluting biologic agents which block cell proliferation, e.g., paclitaxel, sirolimus, zirolimus, etc.

In other variations, the expander may be coated with or integrate a layer of material which is hydrophobic or superhydrophobic to further discourage fluid exposure of the expander structure to the urine.

Additionally, the expander may also optionally incorporate various mechanisms for emitting any number of forms of energy or the expanders may be used along with various forms of energy which may be administered separately from the expander. For instance, an expander may administer thermal, electrical, microwave, mechanical, etc. energy to the tissue prior to, during, or after deployment of the expander. The source of the energy may be controlled or delivered externally from the patient body, e.g., via ultrasound or magnetic induction.

Additionally, the expanders may be comprised of any number of biocompatible materials, e.g., metals such as Nitinol or stainless steel, plastics, polymers such as silicone, PET, PTFE, etc., etc., which may be utilized in any number of combinations either within a single expander or between several expanders utilized together. While the expander structure may remain within the tissue walls, portions of the expander or the entire expander structure may be optionally configured to biodegrade or bioabsorb over a specified period of time.

In the event that the expander is comprised of a shape memory alloy such as Nitinol, the expander may be configured to have a predetermined diameter and shape. Prior to and during deployment of the expander within the body lumen, the expander may have a collapsed low-profile configuration suitable for uninhibited delivery into the body lumen. Once the expander has been desirably positioned, the expander may be allowed to self-expand into its deployment configuration as it heats to body temperature, e.g., above 30° C. Alternatively, the expander may be actuated to expand into its deployed configuration where it may then fully expand into deployment. In yet additional variations, a heating or cooling element (e.g., heated or cooled liquid or gas, Peltier junction, etc.) may be deployed with a deployment instrument or separate from the expander to adjustably heat or cool the expander to allow for its re-configuration into its deployed configuration.

The urethral expanders may be configured in a number of different embodiments. With any of the different configurations, a single expander (e.g., having one or more ring-like structures or coils) may be deployed within the lumen or several expanders (e.g., 2 to 20 expanders or more) may be deployed adjacent to one another. In other variations, 2 to 4 expanders (e.g., each expander having one or more ring-like structures or coils) may be deployed while in other variations, 3 expanders may be deployed. When deployed, sufficient spacing (e.g., spacing may range anywhere from, e.g., 3 mm to 20 mm) may be provided between adjacent expanders along the prostatic urethra PU to allow for the invagination of the expanders by the tissue wall. The spacing between the expanders shall be optimized to permanently relieve the obstruction while minimizing the total amount of material placed in the urethra to avoid tissue irritation or encrustation. Alternatively, the expanders may be deployed to overlap with respect to adjacent expanders. These several expanders may be deployed in a sequential or connected manner within the prostatic urethra PU that facilitates an open passageway from the bladder BL to the membranous urethra MU.

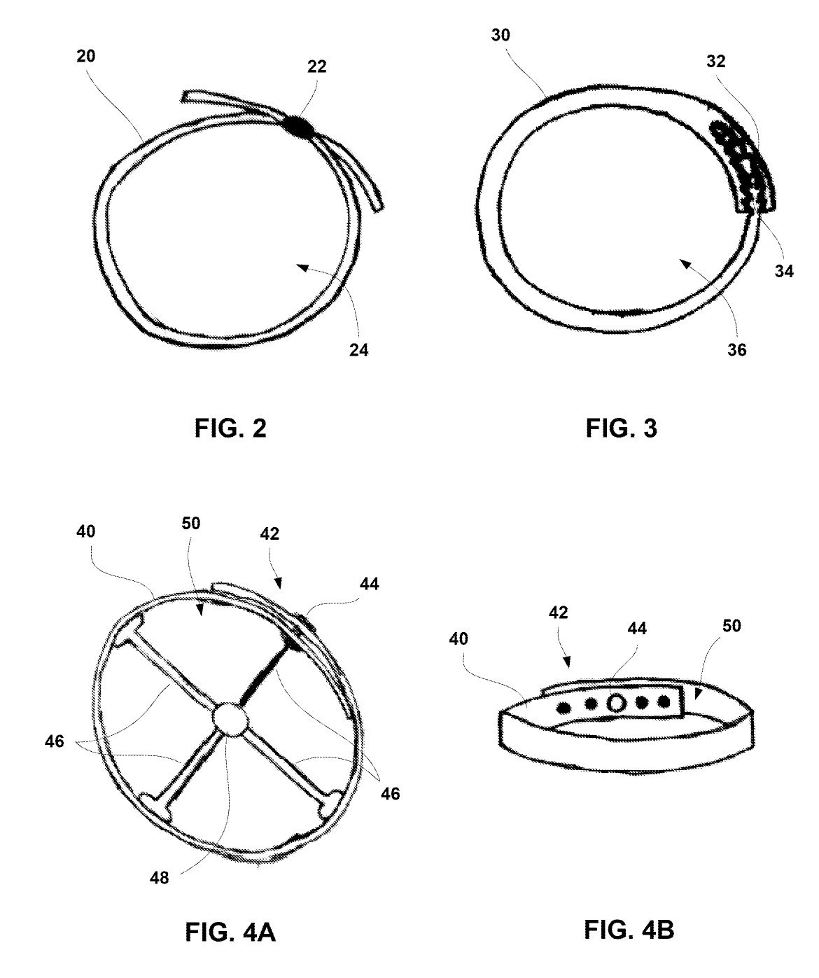

One variation is shown in the top view of FIG. 2 which shows an expander 20 which defines an opening 24 for passage of urine therethrough. The expander 20 may be configured to be adjustably sized either prior to deployment or during deployment via an adjustable locking mechanism 22 which may allow for the sliding release and securement of the free ends of the expander member. The locking mechanism 22 with adjustable ends may allow for the diameter of the expander 20 to be adjusted between a specified minimum and maximum length, as described herein. FIG. 3 shows a top view of another variation of an expander 30 which defines an opening 36 and having a first end 32 which defines an opening for receiving a second end 34. The first end 32 may define a receiving channel having a number of projections for receiving the second end 34 which may define one or more complementary projections such that the second end 34 may be adjustably slid into the first end 32 to allow for a ratcheted adjustment and securement between the ends.

Another variation is shown in the top and perspective views of FIGS. 4A and 4B which illustrate an expander 40 which defines an opening 50 therethrough. In this variation, the ends of the expander 40 may be secured to one another via a pin or lock 44 which may extend through openings 42 defined in each of the ends. This variation may also incorporate one or more struts 46 which may be coupled at a hub 48 and extend through the opening 50 to support the expanded configuration of the expander 40 once deployed.

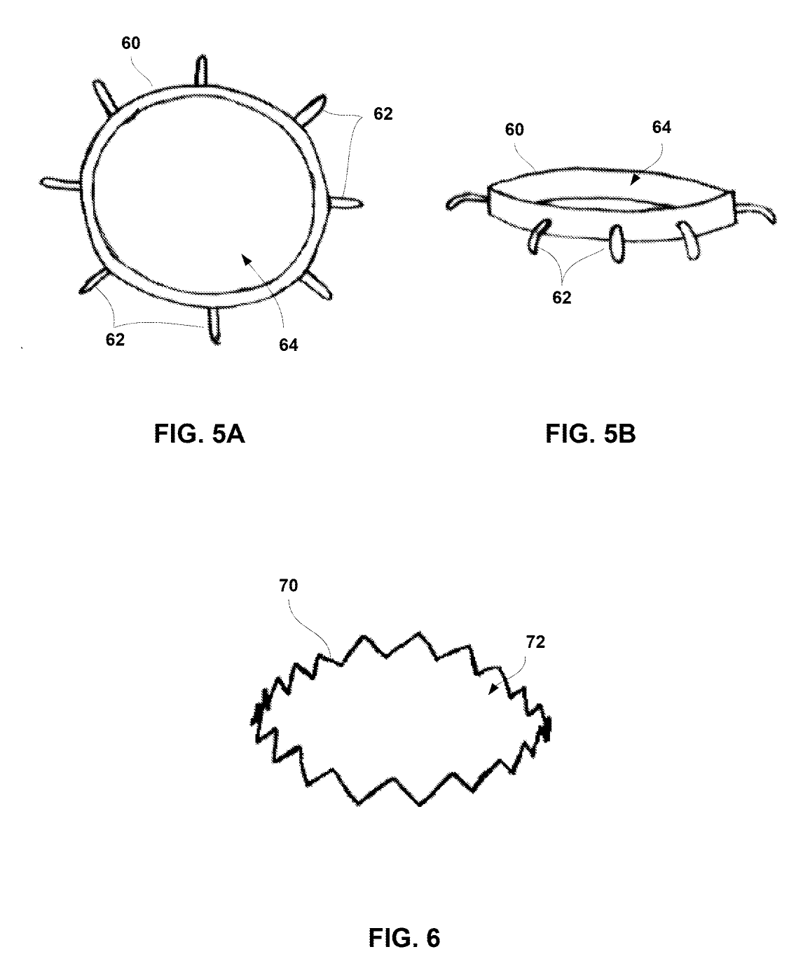

Optionally, the expander structure may also incorporate an anchoring mechanism to facilitate securement of the structure to the surrounding tissue and to prevent its migration when in use. One variation of the anchoring mechanism may be seen in the top and perspective view of FIGS. 5A and 5B which show an expander 60 defining an opening 64 and incorporating one or more tissue anchors 62 which extend radially from an outer surface of the expander 60. While a single tissue anchor 62 may be used, multiple anchors 62 may also be utilized in a uniform or arbitrary pattern along the outer surface of the expander 60. Moreover, such anchors may be configured in any number of shapes or patterns or projections which help to prevent expander migration, e.g., hook-shaped, barb-shaped, V-shaped, etc. Additionally, the anchors may not only be configured to extend radially form the expander 60, they may be configured to extend in alternate directions as well. During delivery of the expander 60 within the body lumen, the anchors may be configured to have a low profile to prevent inadvertent engagement with the tissue wall but when the expander 60 is deployed against the tissue wall, the anchors may be reconfigured, e.g., rotated or repositioned, to face the vessel wall upon deployment. The anchors may also be composed of the same material as the expander 60 or they may be composed of another biocompatible material. While the anchors are illustrated in this particular example, such anchors may be optionally incorporated into any of the other variations of expanders as described herein.

In yet another variation of an expander structure, the expander 70 may be configured into a collapsible structure forming a pattern, e.g., a zig-zag, coiled, sinusoidal, or other pattern, defining an opening 72 which may facilitate the collapse and expansion of the expander 70 along its circumference, as shown in the perspective view of FIG. 6. In this variation, the expander 70 may be expanded to the desired diameter which may then resist its collapse.

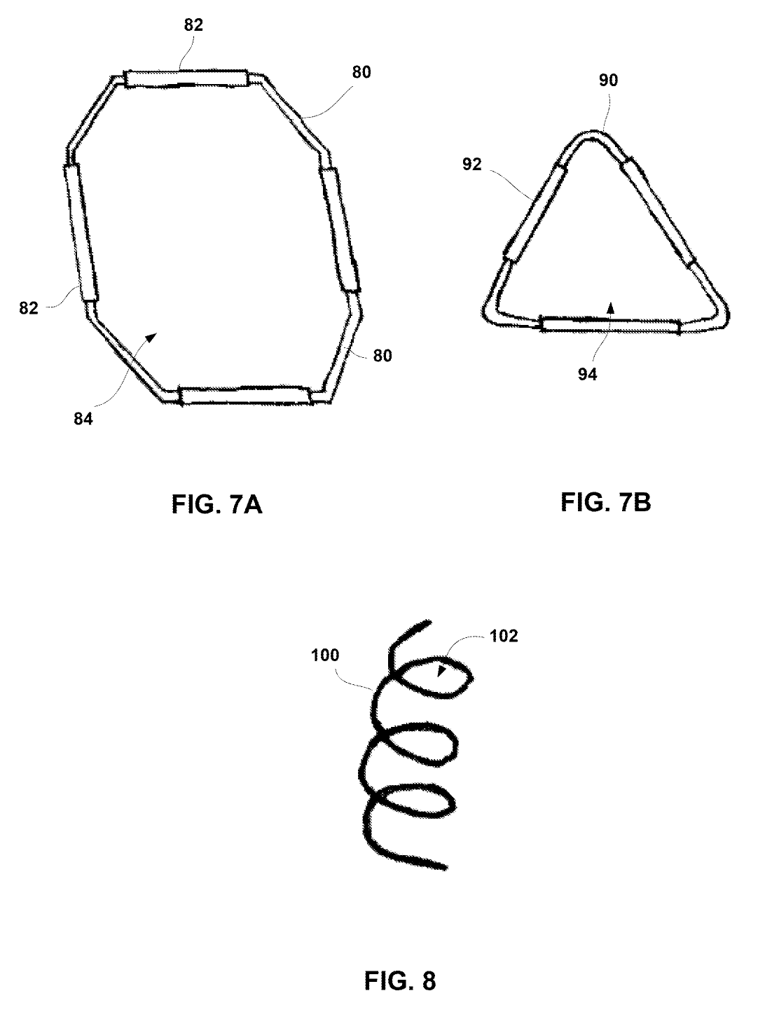

Another variation of the expander structure may be seen in the top views of FIGS. 7A and 7B which illustrate structures which are formed by telescoping elements comprised of separate sections which may be assembled into any number of polygonal shapes (e.g., square, rectangular, circular, triangular, elliptical, etc.). FIG. 7A shows one variation of an expander assembly 80 which defines an opening 84. The expander assembly 80 may form an octagonal shape (although other shapes are possible) where individual receiving sections 82 may form female receiving channels along one or both ends for translatably receiving sections 80 which may form male ends for insertion into the corresponding female channels of alternating receiving sections 82. The connections between the male and female ends may be configured to be uni-directional (e.g., ratcheting mechanisms within the receiving sections 82) so as to provide an expander structure which may be expanded to a deployed shape against the tissue walls which then resists collapse. FIG. 7B shows another variation of an expander assembly 90 which defines an opening 94. In this variation, the assembly 90 may form a triangular shape having receiving sections 92 between adjustable male structures. Additionally, the assemblies may include any number of anchoring aspects along the external surfaces of the structures (as described herein) to prevent migration of the structures after deployment.

In yet another variation, FIG. 8 shows another expander 100 which is configured into a helical or spiral configuration which defines a lumen 102 through the structure. This expander 100 may be comprised of a single unitary structure which is maintained in a low-profile configuration for delivery within the urethra. Once deployed, the expander 100 may reconfigure into its helical or spiral configuration against the tissue walls. Materials such as superelastic or shape memory alloys like Nitinol may be suitable for such a structure although other materials (as described herein) may also be utilized.

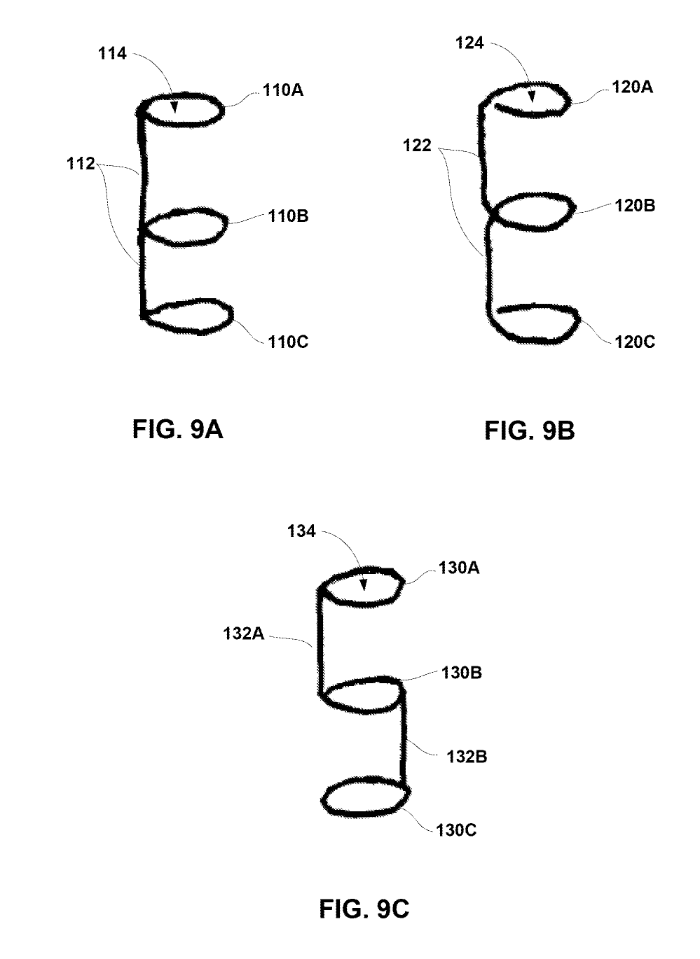

Other variations may be further seen in the perspective views of FIGS. 9A to 9C. FIG. 9A shows one variation of a single expander structure assembly formed of separate ring-like structures 110A, 110B, 110C which are aligned with respect to one another to define a lumen 114 and which are coupled to one another via connector elements 112. The structure may be formed of a wire, ribbon, or other similar structure having various cross-sectional profiles. For instance, the cross-section of the wire or ribbon may form any number of profile shapes, e.g., circular, elliptical, rectangular, square, trapezoidal, etc. Co-linearly aligned connector elements 112 are shown coupling the ring-like structures 110A, 110B, 110C along a tangential portion such that the ring-like structures 110A, 110B, 110C are non-planar and parallel with respect to one another. However, the ring-like structures 110A, 110B, 110C may be angled in a non-parallel manner with respect to one another in other variations. The ring-like structures 110A, 110B, 110C may be formed as individual structures which are then connected to the connector elements 112 although in other variations, the entire structure may be formed of a single element.

Furthermore, although these ring-like structures are formed as a single expander assembly, the spacing between the ring-like structures may be similar to the deployed spacing between the individual expanders described herein. The distance between the structures may accordingly range anywhere from, e.g., 3 mm to 20 mm apart from one another to facilitate tissue epithelialization of the structure. Moreover, the distance between adjacent ring-like structures may be applied to any of the various embodiments described herein.

Moreover, the variation shown has three ring-like structures 110A, 110B, 110C for positioning along each of the three main lobes of the prostate PR so that the ring-like structures 110A, 110B, 110C may become invaginated within the tissues of their respective lobes. Although alternative variations may include a structure having fewer than three or more than three ring-like structures as well as structures which are non-circular in shape but which may include any number of other shapes, as described herein.

FIG. 9B shows another variation where three ring-like structures 120A, 120B, 120C may define a lumen 124 and which may be formed by a single structure which also connects each of the ring-like structures 120A, 120B, 120C via connector elements 122. A single element such as a wire, ribbon, or other similar structure may be used to form a unitary structure where the ring-like structures 120A, 120B, 120C are formed by looping the element.

FIG. 9C shows a perspective view of yet another variation where the ring-like structures 130A, 130B, 130C may define a lumen 134 and which are connected by connector elements 132A, 132B. In this variation, the connector elements 132A, 132B may be parallel to one another but positioned along opposite ends of the ring-like structures 130A, 130B, 130C. In alternative variations, the connector elements 132A, 132B may be positioned at other angles relative to one another instead of being positioned at opposite ends.

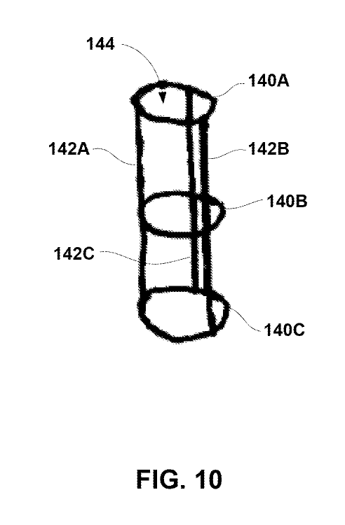

FIG. 10 shows a perspective view of yet another variation where the ring-like structures 140A, 140B, 140C may define a lumen 144 but in this variation, several connector elements 142A, 142B, 142C may be arranged in parallel with respect to one another to couple the ring-like structures to one another. Although shown as being uniformly spaced apart from one another in this variation, the connector elements 142A, 142B, 142C may be spaced at arbitrary positions and may also number two connector elements or more than three connector elements.

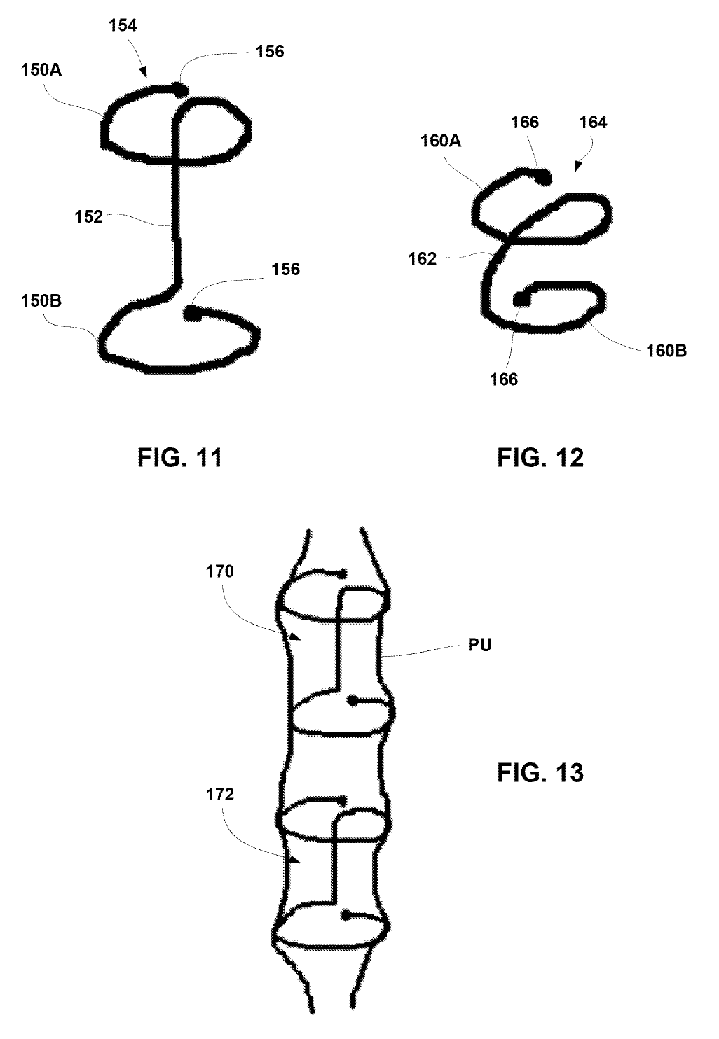

FIG. 11 shows a perspective view of yet another variation of an expander assembly having two ring-like structures 150A, 150B which define a lumen 154 and are connected by a single connector element 152 which is perpendicularly oriented with respect to the structures 150A, 150B. When deployed, the ring-like structures 150A, 150B may be transversely oriented relative to the urethra lumen while the connector element 152 is longitudinally aligned with respect to the urethra lumen. In this variation, the expander assembly may be formed from a single, uniform element which is configured into the expander assembly. Hence, the terminal ends of the ring-like structures 150A, 150B may be enlarged to present a blunt and atraumatic tip 156 to the surrounding tissue to prevent any perforations.

FIG. 12 shows a perspective view of yet another variation of an expander assembly having two ring-like structures 160A, 160B which define a lumen 164 and where the structures 160A, 160B are connected by a single connector element 162. The element 162 may be curved relative to the structures 160A, 160B such that the assembly presents a continuously curved or arcuate structure when deployed. The terminal ends of the ring-like structures 160A, 160B may also be enlarged to present blunt and atraumatic tips 166.

The blunt and atraumatic tips on the ring-like structure may be optionally incorporated into any of the expander embodiments described herein, as practicable. For instance, the embodiments shown in any of the figures such as FIGS. 1C, 8, and 9A to 9C may optionally incorporate such tips.

FIG. 13 shows a perspective view of one example of the expander assembly of FIG. 11 deployed within the prostatic urethra PU. In this example, each of the single expanding assemblies 170, 172 may each have two ring-like structures and the expanding assemblies 170, 172 may be deployed adjacent within the urethra to form a continuous structure which may maintain the urethra in an open configuration. As previously described, each of the assemblies 170, 172 may have one or more ring-like structures or coils, e.g., anywhere from two to four, for deployment. Additionally, the spacing between the ring-like structures in expander assembly 170 as well as between the ring-like structure of an adjacent assembly 172 may range anywhere from, e.g., 3 mm to 20 mm apart from one another to facilitate tissue epithelialization of the structure.

Each of the expander configurations shown utilizing three structures (e.g., for positioning against each of the three main lobes of the prostate PR) may be altered in other variations to include fewer than three structures or more than three structures. Moreover, the relative spacing between the structures may also be varied and the structures themselves may be angled with respect to one another as well. Additionally, any of the structures may optionally incorporate any of the surface modifications such as anchoring mechanisms or various coatings or coverings, as described herein, in any number of various combinations.

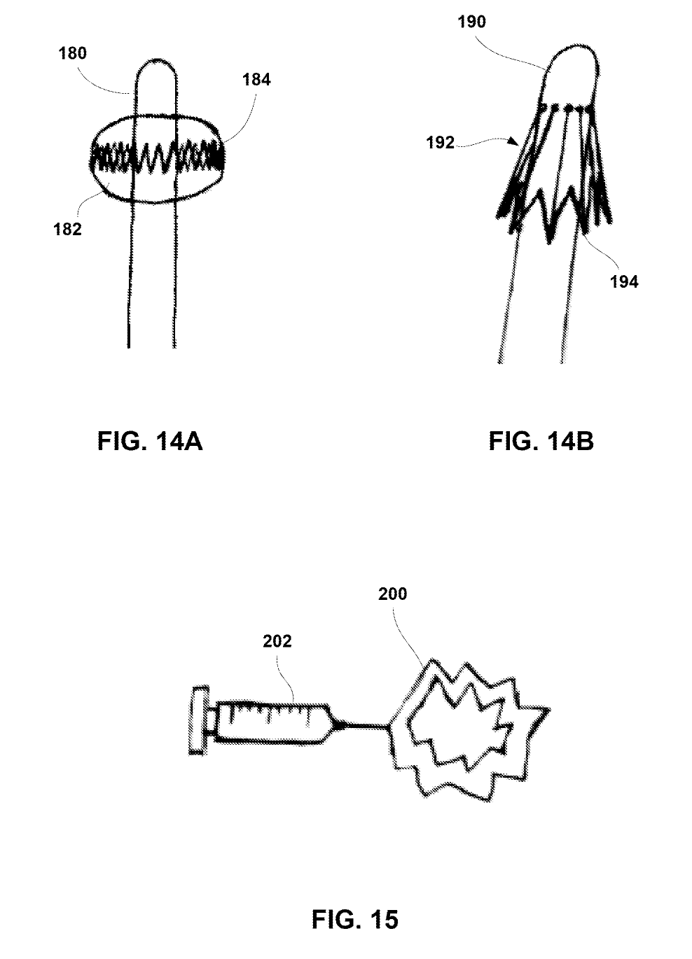

In deploying any of the various expander assemblies described herein, various instruments may be employed. One example is shown in the side view of FIG. 14A which illustrates a delivery and deployment instrument having an elongate shaft 180 having a diameter and a length suitable for insertion into the urethra of the patient. The shaft 180 may be formed with a rigid length or partially flexible length so long as the shaft 180 may be advanced, e.g., within the urethral opening UO and at least partially into the prostatic urethra PU. The shaft 180 may have an inflatable member such as a balloon 182 positioned near or at the distal end of the shaft 180 which may have one or more expanders 184 positioned upon the balloon 182 in a low profile for intra-luminal delivery. Once the balloon 182 has been suitable positioned within the body lumen, the balloon 182 may be expanded to deploy the one or more expanders 184 into contact against the lumen wall. Once deployed, the balloon 182 may be collapsed for removal from the patient body. FIG. 14B shows a side view of another variation of a shaft 190 having an umbrella-like expansion mechanism 192 for deploying one or more expanders 194 against the tissue walls. FIG. 15 shows a side view of yet another variation of a deployment instrument having an inflation reservoir 202 and a tubular expander 200 which may support one or more expanders upon the outer surface of the expander 200.

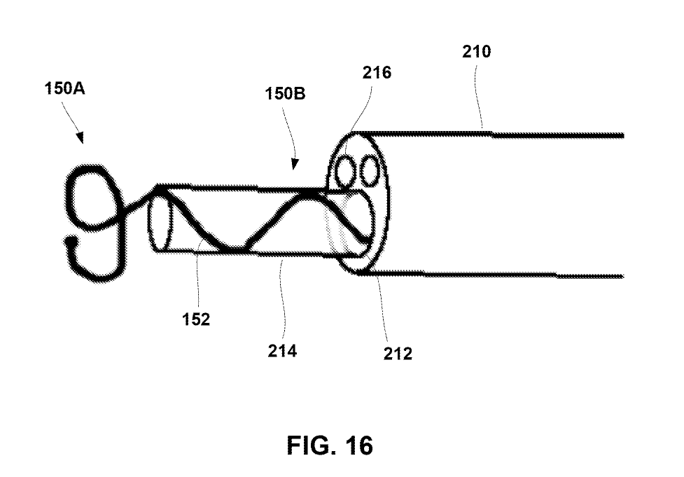

In other variations, as shown in the side view of FIG. 16, the deployment instrument 210 such as an endoscope or cystoscope may define a delivery lumen 212 (e.g., less than 2 mm in diameter) within which the one or more expanders may be positioned while maintained in a low-profile configuration. A pusher or release mechanism may be used to urge the one or more expanders from the delivery lumen 212 in a controlled release once the instrument 210 has been suitably positioned. Moreover, any of the deployment instruments may incorporate any number of steerable components for facilitating advancement of the device within the body lumen. A separate sheath 214 may be advanced or extended through the delivery lumen 212 to help maintain the expander in a low-profile configuration, as shown by the expander assembly and ring-like structures 150A, 150B as well as connecting element 152 shown in their low-profile delivery configuration. The sheath 214 may be composed of various materials (e.g., PEEK, polyimide, stainless steel, Nitinol, etc.) and may be optimized for strength, maximum inner diameter, and flexibility so as to not inhibit the advancement and/or steering of the instrument 210. One or several expander assemblies may be positioned within the instrument 210 for sequential deployment such multiple expanders may be deployed in a single procedure.

Once the expander assembly has been delivered from the instrument 210, the expander may reconfigure itself through its superelastic or shape memory properties into its deployed and expanded configuration into contact against the tissue walls. Alternatively, other actuation mechanisms, as described herein, may be used to reconfigure or facilitate reconfiguration of the expander.

The deployment instrument 210 may be advanced and positioned within the body lumen using an intraluminal visualization system, for example, an endoscope or cystoscope having an imager 216 which may be integrated with the deployment instrument 210 or separated. The visualization system may be utilized either before or during the deployment procedure. In other variations, an external imaging modality (e.g., ultrasound, computed tomography, magnetic resonance imaging, etc.) may be used in combination with the deployment instrument (and/or intraluminal visualization system). In other variations, a balloon or other positioning reference system may be placed, e.g., within the bladder BL or elsewhere to ensure proper anatomic positioning of the expanders.

The system may also optionally include apparatus and methods for adjustment and/or retrieval of the one or more expanders. One embodiment of this aspect of the system may include a grasper mechanism (e.g., standard grasper tools may be used). Other variations may also include heating or cooling elements to adjust the device to facilitate adjustment and/or removal.

The applications of the disclosed invention discussed above are not limited to certain treatments or regions of the body, but may include any number of other treatments and areas of the body. Modification of the above-described methods and devices for carrying out the invention, and variations of aspects of the invention that are obvious to those of skill in the arts are intended to be within the scope of this disclosure. Moreover, various combinations of aspects between examples are also contemplated and are considered to be within the scope of this disclosure as well.

1. An apparatus for maintaining patency of a prostatic urethra lumen, comprising:
an expander structure defining an opening and configured for contact against a tissue lining of the prostatic urethra lumen,
wherein the expander structure is a unitary structure having ring-like structures formed by looping the expander structure, the ring-like structures being non-coplanar and coupled by tangential portions of the expander structure,
wherein the ring-like structures of the expander structure define a deployment diameter which is sized to be 8 mm or greater such that the deployment diameter is relatively larger than an expanded diameter of the prostatic urethra lumen, and
wherein the expander structure has a height which is configured to be invaginated within the tissue lining of the prostatic urethra lumen while maintaining patency of the prostatic urethra lumen.

2. The apparatus of claim 1 wherein the expander structure comprises an adjustment mechanism for altering a diameter of the structure.

3. The apparatus of claim 1 further comprising one or more anchoring mechanisms defined along the expander structure for facilitating tissue engagement.

4. The apparatus of claim 1 wherein the height of the expander structure is between 0.2 mm to 1.5 mm.

5. The apparatus of claim 1 wherein the expander structure has a width between 0.2 mm to 1.5 mm.

6. The apparatus of claim 1 wherein the expander structure is comprised of two or more ring-like structures spaced apart from one another.

7. The apparatus of claim 6 wherein the tangential portions comprise one or more connecting elements coupling the two or more ring-like structures to one another.

8. The apparatus of claim 1 further comprising one or more additional expander structures, wherein each of the expander structures are spaced 3 mm to 20 mm apart from one another to facilitate tissue invagination.

9. A method for maintaining patency of a prostatic urethra lumen, comprising:
advancing at least one expander structure into the prostatic urethra lumen which is to be maintained in an open configuration, wherein the expander structure is a unitary structure having ring-like structures formed by looping the expander structure, the ring-like structures being non-coplanar and coupled by tangential portions of the expander structure;
deploying the at least one expander structure against a tissue lining of the prostatic urethra lumen such that the ring-like structures of the expander structure define a deployment diameter sized to be 8 mm or greater such that the deployment diameter is relatively larger than an expanded diameter of the prostatic urethra lumen; and
maintaining a position of the at least one expander structure within the prostatic urethra lumen such that the at least one expander structure is partially or completely invaginated within the tissue lining of the prostatic urethra lumen while maintaining patency of the prostatic urethra lumen.

10. The method of claim 9 wherein deploying the at least one expander structure comprises altering the deployment diameter of the expander structure.

11. The method of claim 9 wherein maintaining a position comprises anchoring the at least one expander structure via one or more anchoring mechanisms defined along, the expander structure for facilitating tissue engagement.

12. The method of claim 9 wherein the at least one expander structure has a height of between 0.2 mm to 1.5 mm.

13. The method of claim 9 wherein the at least one expander structure has a width of between 0.2 mm to 1.5 mm.

14. The method of claim 9 wherein deploying the at least one expander structure comprises deploying at least two or more ring-like structures spaced apart from one another.

15. The method of claim 14 further comprising one or more connecting elements coupling the two or more ring-like structures to one another.

16. The method of claim 9 further comprising deploying one or more additional expander structures against the tissue lining, wherein each of the expander structures are spaced 3 mm to 20 mm apart from one another to facilitate tissue invagination.

17. The method of claim 9 wherein deploying comprises reconfiguring the at least one expander structure from a low-profile configuration within the prostatic urethra of a patient body.

18. The method of claim 17 wherein deploying further comprises deploying the at least one expander structure against one or more lobes of a prostate within the prostatic urethra.

19. A method for maintaining patency of a body lumen, comprising:
advancing at least one expander structure into the body lumen which is to be maintained in an open configuration;
deploying the at least one expander structure from a low-profile configuration into a deployed configuration against a tissue lining of the body lumen such that the expander structure forms one or more ring-like structures coupled by tangential portions of the expander structure, where the ring-like structures are formed by looping the expander structure to define a deployment diameter which is relatively larger than an expanded diameter of the body lumen; and
maintaining a position of the one or more ring-like structures within the body lumen until the at least one expander structure is partially or completely invaginated within the tissue lining of the body lumen.

20. The method of claim 19 wherein the deployment diameter is sized to be 8 mm or greater.

21. The method of claim 19 wherein the body lumen comprises a prostatic urethra lumen.
